(19) United States
(12) Patent Application Publication
(10) Pub. No.: US 2004/0052855 A1
(43) Pub. Date: Mar. 18, 2004

(54) Microparticles of biodegradable polymer encapsulating a biologically active substance and sustained release pharmaceutical formulations containing same

(76) Inventors: Evelyne Vuaridel, Nyon (CH); Piero Orsolini, Martigny (CH)

Correspondence Address: NIXON & VANDERHYE, PC, 1100 N GLEBE ROAD, 8TH FLOOR, ARLINGTON, VA, 22201-4714, US

(21) Appl. No.: 10/250,857

(22) PCT Filed: Jan. 28, 2002
(86) PCT No.: PCT/CH02/00048

(51) Int. Cl. A61K 9/16; A61K 9/50; B01J 13/02; B01J 13/04

(52) U.S. Cl. 424/490; 264/4.1

(57) ABSTRACT

The present invention relates to novel microparticles of biodegradable polymer encapsulating a water-soluble or water-insoluble biologically active substance, a method for preparing same and a burst free sustained release pharmaceutical formulation comprising those microparticles.

The present invention relates to novel microparticles of biodegradable polymer encapsulating a water-soluble or water-insoluble biologically active substance, a method for preparing same and sustained release pharmaceutical formulation comprising those microparticles.

Many different methods of preparation of microparticles are described in the literature (Herrmann et al., European Journal of Pharmaceutics and Biopharmaceutics 45 (1998) 75-82). The methods presently used for the preparation of microparticles from hydrophobic polymers generally are organic phase separation and solvent removal techniques.

The solvent removal techniques can be divided into solvent evaporation, solvent extraction, spray drying and supercritical fluid technology. In solvent evaporation or solvent extraction techniques, a drug containing organic polymer solution is emulsified into an aqueous or another organic solution. The drug is dissolved, dispersed or emulsified in the inner organic polymer solution.

These solvent removal techniques for production of microspheres by evaporation or extraction necessitate the step of preparing a stable emulsion of organic droplets before solvent removal. The size and characteristics of the final microspheres depend on this step during which a stable emulsion in the presence of the solvent is a prerequisite. The proportions of organic solvent and aqueous phase in the solvent removal methods are carefully maintained so as to control the solvent migration in the aqueous phase. Below a certain ratio organic solvent/aqueous phase, the formation of droplets is not possible any more (see H. Sah, “Microencapsulation techniques using ethyl acetate as a dispersed solvent: effects of its extraction rate on the characteristics of PLGA microspheres,” Journal of controlled release, 47 (3) 1997, 233-245). In some methods, solvent is even added to the aqueous phase in order to saturate it and to prevent the solvent migration during the formation of the primary emulsion.

Several related patents and published applications describe various aspects of these processes.

EP 0 052 105 B2 (Syntex) describes a microcapsule prepared by the phase separation technique using a coacervation agent such as mineral oils and vegetable oils.

EP 0 145 240 B1 (Takeda) discloses a method for encapsulating a water-soluble compound by thickening the inner phase of a W/O emulsion, building a W/O/W and subjecting the emulsion to an “in water drying” process. This method brings different drawbacks such as: the necessity of using a thickening agent to retain the drug, and the multi-step procedure including two emulsification steps and the “in water drying” step.

EP 0 190 833 B1 (Takeda) describes a method for encapsulating a water-soluble drug in microcapsules by increasing the viscosity of a primary W/O emulsion to 150-5,000 cp (by the procedure of increasing the polymer concentration in the organic phase or by adjusting the temperatures) prior to formation of a second W/O/W emulsion which is then subjected to “in water drying”. The drawbacks of this procedure are the complexity of the necessary steps, including formation of two emulsions (W/O and W/O/W) one after the other, and the step of “in-water drying”.

U.S. Pat. No. 5,407,609 (Tice/SRI) describes a microencapsulation process for highly water-soluble agents. This process involves the distinct steps of forming a primary O/W emulsion, the external aqueous phase being preferably saturated with polymer solvent. This O/W emulsion is then poured to a large volume of extraction medium in order to extract immediately the solvent. The drawback of this method is that the O/W emulsion is formed in the presence of the organic solvent in a small volume. The solvent is subsequently removed by extraction in a large aqueous volume. The polymeric droplets are prevented to harden in the primary emulsion, allowing the migration of the drug into the external phase.

WO 95/11008 (Genentech) describes a method for the encapsulation of adjuvants into microspheres. The process comprises the three distinct steps of preparing a primary W/O emulsion, followed by the production of a W/O/W and finally the hardening of the microspheres by extraction of the solvent. As already mentioned above, the drawback of such a method is the complication due to a multi-step procedure separating droplet production from solvent elimination.

EP 0 779 072 A1 (Takeda) describes an “in-water drying” method used for the removal of solvent after production of a W/O/W or a O/W emulsion. It is mentioned that the O/W method is preferable for active substances insoluble or sparingly soluble in water.

WO 00/62761 discloses a method for the preparation of microparticles encapsulating water-soluble biologically active substances with an extremely high encapsulation rate thanks to the optimal reduction of diffusion for the substance to be encapsulated. That method comprises the steps of first incorporating a biodegradable polymer in an organic liquid phase comprising at least one organic non-water miscible solvent, then pouring said organic phase being into an aqueous liquid phase having a volume which is sufficient to dissolve said organic solvent, said aqueous phase containing a surfactant, and homogenizing the resulting organic/aqueous phase in order to perform in one single step the microparticle formation and the organic solvent removal. The microparticles are collected at the end of the homogenization step by filtration and then vacuum dried at room temperature (see Examples 1 to 6).

The applicant has now surprisingly found that performing the sequence of steps of the method disclosed in WO 00/62761 with the difference that microparticles collected at the end of the homogenization step are suspended without vacuum-drying thereof in a lyophilisation medium, yields novel microparticles of compartmentalized structure: they are non porous microparticles of irregular spheroidal shapes wherein pocket microparticles contain microparticles of smaller size, the active substance being evenly distributed within the polymer matrix.

Probably due to that compartmentalized structure and/or the low level diffusion external to the microparticles during the preparation process thereof, those microparticles have the advantageous property of releasing the active substance in a regular and slow manner. When the core loading of active substance is below a threshold value, those microparticles show no or a very low burst, probably due to a molecular dispersion of the active principle within the polymer matrix. Other novel microparticles of similar structure having those advantageous properties are obtained when performing the same sequence of steps with a water-insoluble biologically active substance.

The invention thus concerns microparticles of biodegradable polymer encapsulating a water-soluble or water-insoluble biologically active substance, wherein pocket microparticles contain microparticles of smaller size, wherein said microparticles are obtainable by a method comprising

(a) pouring an organic liquid phase comprising, in a dissolved state the biodegradable polymer and in a uniformly distributed state the biologically active substance, in a non-water miscible organic solvent showing a low solubility in water, into an aqueous liquid phase of sufficient volume to dissolve said organic solvent, said aqueous phase containing a surfactant, and homogenizing the resulting organic/aqueous phase, thereby forming a suspension of microparticles, and

(b) filtering the suspension obtained in (a), optionally washing the microparticles with water, suspending the microparticles without vacuum-drying thereof in a lyophilization medium and freeze-drying.

When the biologically active substance is water-soluble, the organic liquid phase may be prepared by dissolving or dispersing that substance in a volume of water, dissolving the biodegradable polymer in a 10 to 100 larger volume of non-water miscible organic solvent showing a low solubility in water, and mixing under vigorous agitation the aqueous and the organic solutions obtained, e.g. by pouring the aqueous solution into the organic solution and homogenizing the mixture at high rotation speed, for example using a Polytron PT 6100 (PT-DA 3020/2TM shaft) at 10 000 to 30 000 rpm.

When the biologically active substance is water-insoluble, the organic liquid phase may be prepared by dissolving that substance together with the biodegradable polymer in a non-water miscible organic solvent showing a low solubility in water.

One of the specific features in the process for preparing the microparticles is that no stable emulsion comprising organic solvent droplets occurs in step (a) when pouring the organic liquid phase into an aqueous liquid phase of sufficient volume to dissolve said organic solvent. Avoiding such a step results in a better retention of the biologically active substance and a direct harvesting of the microparticles after their formation.

Because the microparticle formation and the solvent removal are done together in one single step in this process, the water-soluble biologically active substance is quickly kept inside the microparticles which have an impermeable wall. Thereby any diffusion of the active substance external to the microparticles is at a low level, the encapsulation rate is very high and the amount of the biologically active substance on the surface of the microparticles is minimal. Hence a release of the active substance in a regular and slow manner. When the core loading is low enough for a very fine dispersion, probably a molecular dispersion, of the active principle within the polymer matrix, those microparticles show no or a very low burst.

Those microparticles with no or a very low burst may show an initial release of the active substance of less than 10% during the first 24 hours, or even below 3% during the first 48 hours.

The organic solvents used in the process of the present invention are non-water miscible solvents showing a low solubility in water such as esters (e.g. ethyl acetate, butyl acetate), halogenated hydrocarbons (e.g. dichloromethane, chloroform, carbon tetrachloride, chloroethane, dichloroethane, trichloroethane), ethers (e.g. ethyl ether, isopropyl ether), aromatic hydrocarbons (e.g. benzene, toluene, xylene), carbonates (e.g. diethyl carbonate), or the like. Although these solvents are generally classified by the person skilled in the art as non-water miscible solvent, they are actually sparingly miscible in water, having a low solubility in water. For instance, for ethyl acetate and dichloromethane, the solubility is respectively 8.70% and 1.32% (by weight) in water at 20-25° C. (see A. K. Doolittle Ed., Properties of individual solvents, in The technology of solvents and plasticizers, chpt. 12. Wiley, N.Y., 1954, pp. 492-742). One of the preferred solvent is ethyl acetate.

The above-mentioned organic solvents can be used alone or in mixtures of two or more different solvents.

The volume of the aqueous liquid phase must be sufficient to dissolve, or extract, the total amount of organic solvent used. If this is not the case, the microparticles cannot be sufficiently hardened. Those “soft” microparticles may therefore melt among each other during the filtration process.

Accordingly, the amount of organic solvent is kept as low as possible to get a viscous organic phase and to minimize the necessary volume of the aqueous phase. In all of the following embodiments, the volume of the aqueous phase is chosen to be capable of dissolving at least the complete amount of organic solvent.

The maximal value of the ratio solvent/water (w/w) in the present invention should therefore preferably be 0.087 and 0.013 for ethyl acetate and dichloromethane respectively. In the examples given below, the ratio ethyl acetate/aqueous phase ranges from 0.007 to 0.06. The encapsulating efficiency improves if the volume of aqueous phase increases.

A surfactant is added to the aqueous phase in order to keep the precipitating biodegradable polymer in fine independent particles. An ideal surfactant gives a viscosity to the aqueous phase that approaches the viscosity of the organic phase.

An electrolyte may also be optionally added to the aqueous solution to create repulsion between the particles and preventing aggregation. As a preferred electrolyte, sodium chloride is used in the aqueous phase and leads to a higher encapsulating efficiency.

The aqueous solution can also be buffered to obtain good pH conditions for the drug concerning stability and release.

When a solvent such as ethyl acetate is used, it has been surprisingly found that the encapsulation efficiency is increased when using cold solutions, by optimizing the solubility of the solvent in water, by reducing the aqueous solubility of the drug, and by slowing down its diffusion. In other words, the present invention achieves the effect of further reducing the already small amount of diffusion of internal particle substances to the exterior.

A water-soluble biologically active substance is dispersed as such or as an aqueous solution into one of the above-mentioned non-miscible organic solvent. In some embodiments of the process, the biologically active substance is present in solid state in the organic phase during the entrapment procedure, thus slowing down the solubilisation into the aqueous liquid phase.

The thus obtained liquid organic phase containing the biologically active substance is used to dissolve the biodegradable polymer.

The appropriate biodegradable polymers comprise poly(lactides), poly(glycolides), copolymers thereof or other biodegradable polymers such as other aliphatic polymers, polycitric acid, polymalic acid, polysuccinates, polyfumarates, polyhydroxybutyrates, polycaprolactones, polycarbonates, polyesteramides, polyanhydrides, poly(amino acids), polyorthoesters, polycyano-acrylates, polyetheresters, poly(dioxanone)s, copolymers of polyethylene glycol (PEG), polyorthoesters, biodegradable polyurethanes, polyphosphazenes.

Other biocompatible polymers are polyacrylic acid, polymethacrylic acid, acrylic acid-methacrylic acid copolymers, dextran stearate, ethylcellulose, acetylcellulose, nitrocellulose, etc. These polymers may be homopolymers or copolymers of two or more monomers, or mixtures of the polymers.

A particularly interesting biodegradable polymer is poly(D-L-lactide-co-glycolide).

The biologically active substance and the polymer can also be incorporated in separate organic phases. The polymer is dissolved in another above-mentioned organic non-water miscible solvent. Preferred solvents include ethyl acetate or dichloromethane. More preferred is when the solvent used to dissolve the polymer is the same solvent as that use for incorporating the biologically active substance. The thus obtained separated organic phases are poured together to form a homogenous organic phase before addition to the aqueous phase.

If the biologically active substance and/or the biodegradable polymer is not or is only slightly soluble in one of the above-mentioned solvent, for instance in the preferred solvent ethyl acetate, a sufficient amount of co-solvents such those comprised among the family of benzyl alcohol, DMSO, DMF, ethyl alcohol, methyl alcohol, acetonitrile and the like, may optionally be used for that purpose.

A better encapsulating efficiency can be achieved by an appropriate setting of the physic chemical parameters such as surfactant capacity, viscosity, temperature, ionic strength, pH and buffering potential during the homogenization of the organic inner phase into the aqueous phase. By carefully adjusting the production parameters, the precipitating polymer can be surprisingly well formed into homogeneously dispersed particles.

Preferably, the amount of solvent used to dissolve the biodegradable polymer is kept to a minimum in order to be soluble as quickly as possible (most preferably at once) in the aqueous phase. If the amount of solvent is high, the amount of aqueous phase has to be too large on a practical point of view.

The concentration of polymer in the organic phase is adjusted to 5-90% (by weight), preferably between about 10 and 50%, depending on the polymer and solvent used.

In the case that the concentration of polymer in the organic solvent is high, the viscosity of this phase, depending on the polymer used, may be increased.

The viscosity of the polymer solution may be comprised between 1000 and 40,000 centipoise (cp) (Brookfield viscosity), more preferably between 2,000 and 30,000 cp, even more preferably between 3,000 and 20,000 cp.

Using solvents like ethyl acetate for dissolving the polymer, the solubility of the solvent in the aqueous phase is increased by lowering the temperature of both the organic and the aqueous phases, accelerating the solvent migration and therefore also the encapsulation rate.

In process of the present invention, the temperature of the organic phase ranges between about −10° C. and 30° C., and preferably between about 0° C. and 10° C. For ethyl acetate, the temperature ranges preferably between about 2° C. and 5° C. The temperature of the polymeric organic phase and the temperature of the aqueous phase are the same or different and are adjusted in order to increase the solubility of the solvent in the aqueous phase.

The obtained organic phase for use as the inner polymer and biologically active substance containing phase is added to a aqueous outer phase under a homogenization procedure to give microparticles.

For the homogenization procedure, a method of creating dispersion is used. This dispersion can be realized for example with any apparatus capable of shaking, mixing, stirring, homogenizing or ultrasonicating.

Different agents influencing the physico-chemical characteristics of the resultant medium may be added. For instance, surfactants, such as for example an anionic surfactant (e.g. sodium oleate, sodium stearate, sodium lauryl sulfate), a nonionic surfactant (e.g. polyoxyethylene-sorbitan fatty acid ester (Tween 80, Tween 60, products available from Atlas Powder Co, U.S.A.), a polyoxyethylene castor oil derivative (HCO-60, HCO-50, products available from Nikko Chemicals, Japan)), polyvinyl pyrrolidone, polyvinyl alcohol, carboxymethyl-cellulose, lecithin or gelatine.

In specific embodiments of the present invention, a surfactant comprised among the family of anionic, non-ionic agents or other agents capable of reducing the surface tension of the polymeric dispersion can be added. Suitably, therefore, are nonionic surfactants such as Tween (for example Tween 80), anionic surfactants, nonionic surfactant like polyvinyl alcohol or others. These surfactants can, in general, be used alone or in combination with other suitable surfactants. The concentration of the surfactant is selected in order to disperse and stabilize the polymer particles, and possibly also to give a viscosity approaching the viscosity of the organic phase.

The preferred concentration of the surfactant in the aqueous phase ranges therefore between about 0.01-50% (by weight), preferably between about 5 and 30%. The viscosity depending on the surfactant used and on its concentration ranges between about 1,000-8,000 cp (Brookfield viscosity), preferably about 3,000-5,000 cp.

Optionally salts comprised among the family of sodium chloride, potassium chloride, carbonates, phosphates and the like can be added to the aqueous phase to adjust ionic strength and to create a Zeta potential between the polymer particles, leading to particle repulsion.

Additional buffering agents may be added to the aqueous phase to maintain a specific pH. So, the internal aqueous phase may be supplemented with a pH regulator for retaining stability or solubility of the biologically active substance, such as carbonic acid, acetic acid, oxalic acid, citric acid, phosphoric acid, hydrochloric acid, sodium hydroxide, arginine, lysine or a salt thereof. The pH of the formulations of this invention is generally about 5 to 8, preferably about 6.5 to 7.5.

The temperature of the aqueous phase can be adjusted to the temperature of the inner organic phase. The temperature range is from about −10° C. to 30° C., more preferably between 0° and 10° C. and even more preferably from between 2° C. and 5° C.

The microparticles of the present invention can be prepared in any desired size, ranging from 1 μm to about 500 μm, by varying the parameters such as polymer type and concentration in the organic phase, volumes and temperature of the organic and aqueous phase, surfactant type and concentration, homogenization time and speed. The mean particle size of the microparticles ranges generally from 10 to 200 μm, more preferably from 20 to 200 μm, even more preferably from 30 to 150 μm.

A number of water-soluble active substances can be encapsulated by the process of the present invention.

Preferably, the encapsulated soluble substance is a peptide, a polypeptide, a protein and their related pharmaceutically acceptable salts. The salt of peptide is suitably a pharmacologically acceptable salt. Such salts include salts formed with inorganic acids (e.g. hydrochloric acid, sulfuric acid, nitric acid), organic acids (e.g. carbonic acid, bicarbonic acid, succinic acid, acetic acid, propionic acid, trifluoroacetic acid) etc. More preferably, the salt of peptide is a salt formed with an organic acid (e.g. carbonic acid, bicarbonic acid, succinic acid, acetic acid, propionic acid, trifluoroacetic acid) with greater preference given to a salt formed with acetic acid. These salts may be mono-, di- or tri-salts.

Examples of water-soluble active substances which can be encapsulated in the microparticles of the present invention include, but are not limited to, peptides, polypeptides and proteins such as luteinizing hormone releasing hormone (LHRH) or derivatives of LHRH comprising agonists or antagonists, melanocyte stimulating hormone (MSH), thyrotropin releasing hormone (TRH), thyroid stimulating hormone (TRH), follicule stimulating hormone (FSH), human chorionic gonadotropin (HCG), parathyroid hormone (PTH), human placental lactogen, somatostatin and derivatives, gastrin, prolactin, adreno-corticotropic hormone (ACTH), growth hormones (GH), growth hormone releasing hormone (GHRH), growth hormone releasing peptide (GHRP), calcitonin, oxytocin, angiotensin, enkephalins, endorphin, enkephalin, kyotorphine, interferons, interleukins, tumor necrosis factor (TNF), erythropoetin (EPO), colony stimulating factors (G-CSF, GM-CSF, M-CSF), thrombopoietin (TPO), platelet derived growth factor, fibroblast growth factors (FGF), nerve growth factors (NGF), insulin like growth factors (IGF), amylin peptides, leptin, RGD peptides, bone morphogenic protein (BMP), substance P, serotonin, GABA, tissue plasminogen activator (TPA), superoxide dismutase (SOD), urokinase, kallikrein, glucagon, human serum albumin, bovine serum albumin, gamma globulin, immunomodulators (EGF, LPS), blood coagulating factor, lysozyme chloride, polymyxin B, colistin, gramicidin, bacitracin and the like.

A number of other unlimiting examples of water-soluble substances or particularly a water-soluble form of the following substances can be encapsulated by the process of the present invention.

These substances comprise for instance anticancer drugs such as actinomycin D, bleomycin, carboplatin, carmustine, chlorambucil, cisplatin, cladribine, cyclophosphamide, cytarabine, dacarbazine, daunorubicin, doxorubicin, estramustine, etoposide, floxuridine, fludarabine, fluorouracil, hexamethylmelamine, hydroxyurea, idarubicin, ifosfamide, asparaginase, lomustine, mechlorethamine, melphalan, mercaptopurine, methotrexate, mithramycin, mitomycin C, mitotane, mitozantrone, oxaliplatine, pentostatin, procarbazine, streptozocin, teniposide, thioguanine, thiopeta, vinblastine, vincristine, an aromatase inhibitor such as Fradrazol or Anastrazol and the like; antibiotics such as tetracyclines, penicillins, sulfisoxazole, ampicillin, cephalosporins, erytromycin, clindamycin, isoniazid, amikacin, chloramphenicol, streptomycin, vancomycin, salvicin and the like.

Other examples of such substances comprise analgesics and antiinflammatory agents include acetaminophen, acetylsalicylic acid, methylprodnisolone, ibuprofen diclofenac sodium, indomethacin sodium, flufenamate sodium, pethidine hydrochloride, levorphanol tartrate, morphine hydrochloride, oxymorphone and the like; anesthetics such as xylocaine and the like; antiulcer agents include metoclopramide, ranitidine hydrochloride, cimetidine hydrochloride, histidine hydrochloride, and the like anorexics such as dexedrine, phendimetrazine tartrate, and the like; antitussives such as noscapine hydrochloride, dihydrocodeine phosphate, ephedrine hydrochloride, terbutaline sulfate, isopreterenol hydrochloride, salbutamol sulfate, and the like; antiepileptics such as acetazolamide sodium, ethosuximide, phenytoin sodium, diazepam and the like; antidepressants such as isocarboxazide, pheneizine sulfate, clomipramine, noxiptilin, imipramine, and the like anticoagulants such as heparin or warfarin, and the like.

Other unlimiting examples comprise sedatives such as chlorpromazine hydrochloride, scopolamine methylbromide, antihistaminics such as diphenhydramine hydrochloride, ketotifen fumarate, chlorpheniramine maleate, methoxy-phenamine hydrochloride and the like.

Other unlimiting examples comprise cardiotonics such as etilefrine hydrochloride, aminophylline and the like; antiasthmatics such as terbutaline sulfate, theophylline, ephedrine, cetirizin and the like; antifungals such as amphotericin B, nystatin, ketoconazole, and the like; antiosteopotic agents such as bisphosphonates, e.g. alendronate, antiarrhytmic agents such as propranolol hydrochloride, alprenolol hydrochloride, bufetolol hydrochloride, oxyprenolol hydrochloride and the like; antitubercular agents such as isoniazid, ethambutol, and the like; hypotensive, diuretic agents such as captopril, ecarazine, mecamylamine hydrochloride, clonidine hydrochloride, bunitrolol hydrochloride and the like; hormones such as prednisolone sodium sulfate, betamethasone sodium phosphate, hexestrol phosphate, dexamethasone sodium sulfate and the like; antigens from bacteria, viruses or cancers, antidiabetics such as glipizide, phenformin hydrochloride, buformin hydrochloride, glymidine sodium, methformin, and the like; cardiovascular agents such as propanolol hydrochloride, nitroglycerin, hydralazine hydrochloride, prazosin hydrochloride and the like; diuretics such as spironolactone, furosemide and the like; and enzymes, nucleic acids, plant extracts, anti-malarials, psychotherapeutics, hemostatic agents, etc.

Examples of water-insoluble biologically active substances which can be encapsulated in the microparticles of the present invention include, but are not limited to, anesthetics such as lidocaine and the like, anorexics such as phendimetrazine, antiarthritics such as methylprednisolone, ibuprofen and the like, antiasmathics such as terbutaline and the like, antibiotics such as sulfisoxazole, cephalosporins, tetracyclines, erythromycin, clindamycin and the like, antifungals such as amphotericin B, nystatin, ketoconazole and the like, antivirals such as acyclovir, amantadine and the like, anticancer agents such as methotrexate, etretinate, aromatase inhibitors such as Exemestane, Formestane, Letrozole, Vorozole, Aminoglutetimide and the like, anticoagulants such as warfarin and the like, anticonvulsants such as phenytoin and the like, antidepressants such as amoxapine and the like, antihistamines such as dephenydramine, chlorpheniramine and the like, hormones such as insulin, progestins, thyroxines, estrogens, corticoids, androgens and the like, tranquilizers such as chlorpromazine, reserpine, chlordiazepoxide and the like, antispasmodics such as Belladonna alkaloids, dicyclomine and the like, vitamins and minerals, cardiovascular agents such as prazosin, nitroglycerin, propanolol, hydralazine, linsidomin, verapamil and the like, peptides and proteins such as LHRH, somatostatin, vasopressin and the like, prostaglandins, nucleic acids, carbohydrates, fats, narcotics such as morphine, codeine and the like, psychotherapeutics, anti-malarials, diuretics such as furosemide, spironolactone and the like, and antiulcer drugs such as ranitidine, cimetidine and the like.

Preferred substances include Tamoxiphen, 4-OH Tamoxiphen, a derivative thereof, a non-soluble LHRH derivative such as Triptorelin pamoate and a non-soluble somatostatin derivative such as Octreotide™, Lanreotide™ or vapreotide pamoate.

The invention also concerns a sustained release pharmaceutical formulation which comprises a suspension of the above microparticles in a pharmaceutically acceptable vehicle. Preferably the initial release of the active substance during the first 24 hours is less than 10%. More preferably the initial release during the first 48 hours is less than 3%.

The invention also relates to a method of preparing the above microparticles, which comprises

(a) pouring an organic liquid phase comprising, in a dissolved state the biodegradable polymer and in a uniformly distributed state the biologically active substance, in a non-water miscible organic solvent showing a low solubility in water, into an aqueous liquid phase of sufficient volume to dissolve said organic solvent, said aqueous phase containing a surfactant, and homogenizing the resulting organic/aqueous phase, thereby forming a suspension of microparticles, and

(b) filt ring the suspension obtained in (a), optionally washing the microparticles with water, suspending the microparticles without vacuum-drying thereof in a lyophilization medium and freeze-drying.

The examples that follow are set forth as an aid in understanding the present invention, and provide some examples of the many embodiments that are potentially available for the present invention. They are not intended to limit the scope of the invention.

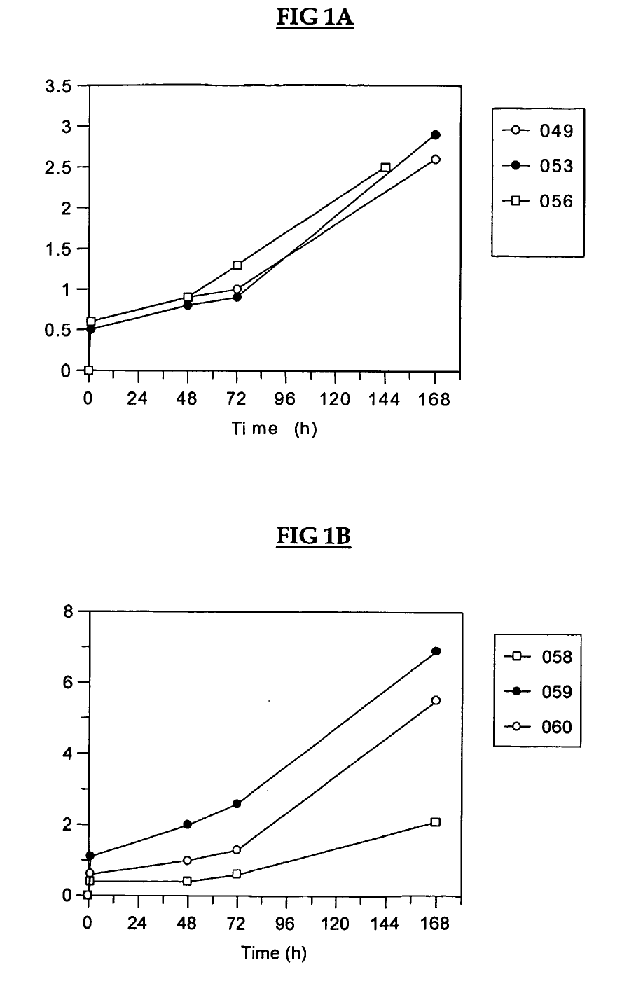

The following description will be better understood by referring to FIGS. 1A, 1B, 2A and 2B, 3, 4 and 5.

FIGS. 1A, 1B, 3 and 4 are curves representing the in vitro release profiles of batches (49, 53 and 56) and (58, 59 and 60) of Triptorelin acetate encapsulating microparticles, batch 4 of Tamoxifen encapsulating microparticles, and batch 5 of 4-OH-Tamoxiphen, respectively.

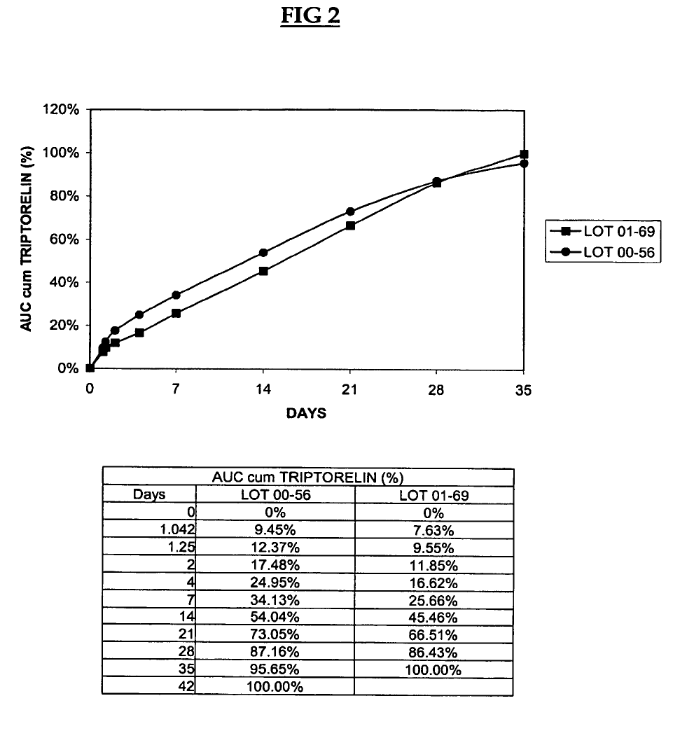

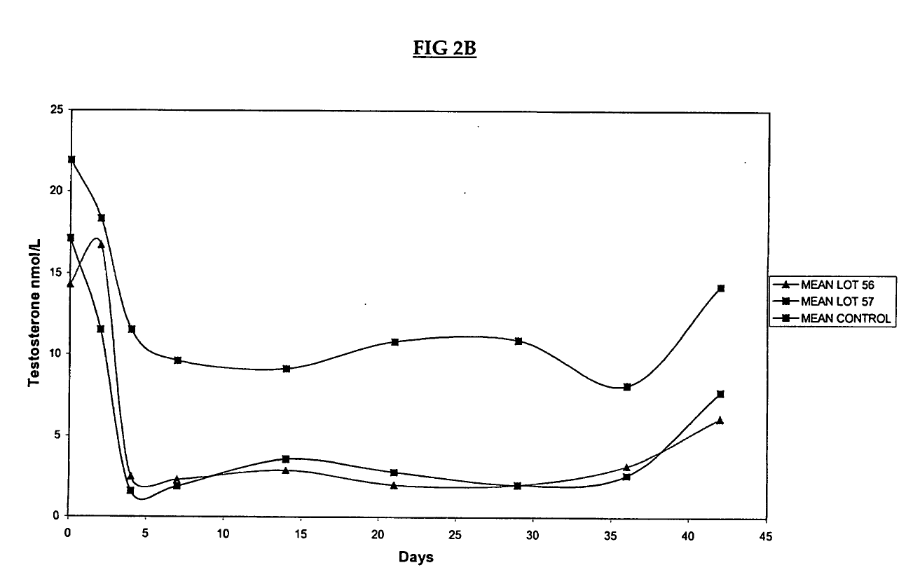

FIG. 2A represents the variation of the cum AUC (cumulated area under the curve) of the Triptorelin acetate level for batches 56 and 69 of Triptorelin acetate as a function of time for rats, injected on day 1 with a suspension of Triptorelin acetate encapsulating microparticles of batches 56 and 69.

FIG. 2B represents the variation of serum testosterone levels as a function of time for rats previously housed close to female rats, injected on day 1 with a suspension of Triptorelin acetate encapsulating microparticles of batches 56 and 57 and non treated rats as a control.

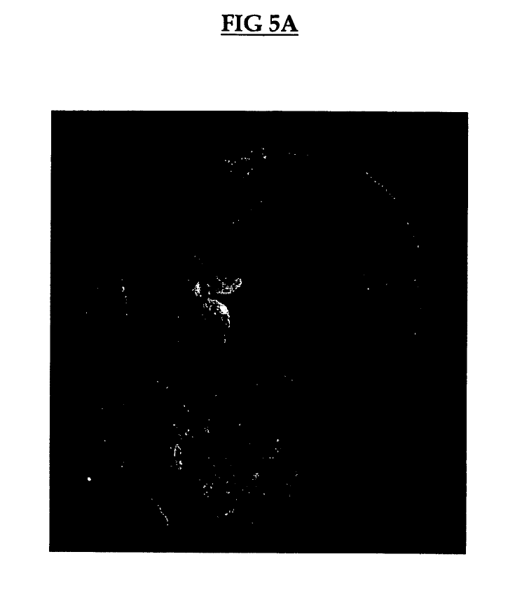

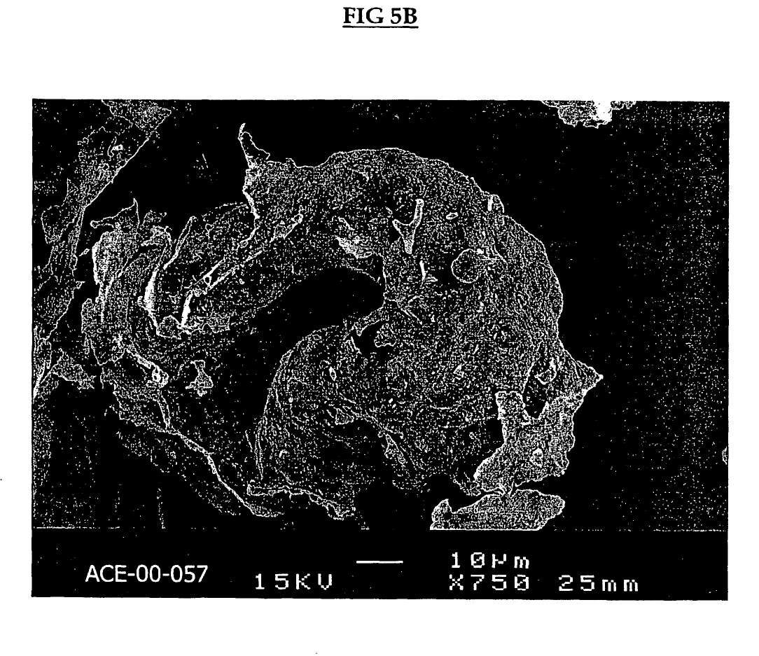

FIGS. 5A and 5B represent scanning electron microscopy photographs of a Triptorelin acetate encapsulating microparticle of batch 57. Those photographs show a primary polymeric non porous pocket microparticle containing secondary polymeric microparticles of smaller size, the active substance being evenly distributed within the polymer matrix.

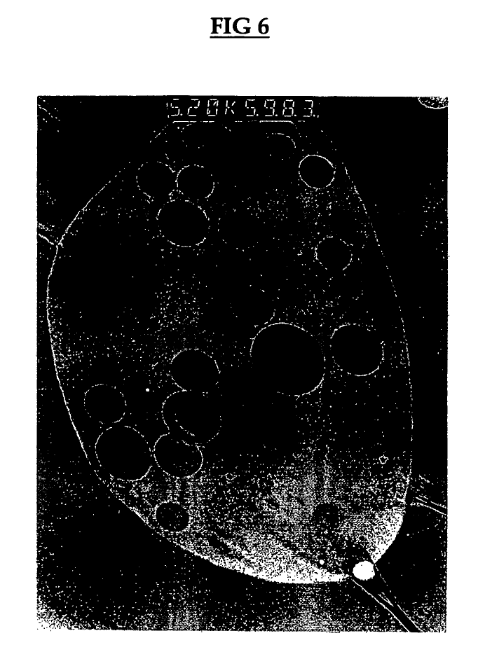

FIG. 6 represents a transmission electron microscopy photograph of a Triptorelin acetate encapsulating microparticle of batch 57 cut in a thin layer. That photograph shows a polymeric non porous pocket microparticle containing secondary microparticles of smaller size.

### EXAMPLE 1

Preparation of Different Batches of Triptorelin Acetate Encapsulating Microparticles

The following sequence of steps was performed for each batch:

1. About 940 mg of D-Trp⁶-LHRH acetate (Triptorelin acetate) were dissolved in 9.4 g of sterile distilled water. This aqueous phase solution was cooled to 4° C.

2. About 25.0 g of poly(D-L-lactide-co-glycolide) (PLGA) with a ratio of lactide to glycolide of 50/50 and a weight average molecular weight of 45,000 were dissolved in 250 g of ethyl acetate at room temperature. This organic phase solution was cooled to 4° C.

3. The aqueous phase solution was poured into the organic phase solution and the mixture was homogenized using a Polytron PT 6100 (PT-DA 3020/2TM shaft) at 20 000 rpm during 2 minutes.

4. This w/o preparation was poured into about 8500 g of aqueous phase containing 20% (w/w) of polyoxyethylene sorbitan fatty acid ester (Tween 80) and possibly 84.4 g of sodium chloride, in a reactor kept at a temperature of 4° C.

5. The homogenization was performed using a Polytron PT 6100 (PT6020/2TM shaft) at 3000-3500 rpm during 5 minutes, thereby forming a suspension of microparticles.

6. The microparticles were collected by filtration and washed with about 9 l of sterile distilled water yielding a bulk of microparticles.

7. The bulk was possibly frozen, kept overnight and thawed.

8. The microparticles were suspended in a lyophilization medium consisting of mannitol, Tween 80 and sodium carboxy-methyl-cellullose using a magnetic rod at 200 rpm, and possibly homogenized using an IKA-T25 homogenizes at 8000 rpm during 20 minutes or at 9500 rpm during 30 minutes (batches 58-60). The suspension was freeze-dried.

The obtained microparticle lyphilisate showed less than 2% residual water.

The entrapment efficiency was measured by UV spectrometry on the bulk of microparticles and by HPLC on the lypholisate and the particle size distribution was determined using a laser granulometer (Mastersizer®, Malvern Instruments).

Batch 49, obtained using a reactor with a conic lower part in steps 4 and 5, no sodium chloride in step 4, no step 7, and no homogenization in step 8, showed a mean particle size of 92.5 μm and an entrapment efficiency of 81.8% on the lyophilisate.

Batch 53, obtained using a reactor with a conic lower part in steps 4 and 5, 84.4 g sodium chloride in step 4, no step 7, and no homogenization in step 8, showed a mean particle size of 96.1 μm and an entrapment efficiency of 75.4% on the lyophilisate.

Batch 56, obtained using a reactor with a conic lower part in steps 4 and 5, no sodium chloride in step 4, and step 7, and homogenization in step 8, showed a mean particle size of 70.0 μm and an entrapment efficiency of 87.7% on the bulk.

Batch 57, obtained using a reactor with a conic lower part in steps 4 and 5, 84.4 g sodium chloride in step 4, no step 7 and homogenization in step 8, showed a mean particle size of 84.3 μm and an entrapment efficiency of 91.7% on the bulk.

Batch 58, obtained using a reactor with a flat bottom in steps 4 and 5, no sodium chloride in step 4, no step 7, and homogenization in step 8, showed a mean particle size of 39.2 μm.

Batch 59, obtained using a reactor with a conic bottom in steps 4 and 5, no sodium chloride in step 4, no step 7, and homogenization in step 8, showed a mean particle size of 59.3 μm.

Batch 60, obtained as batch 59 but with a different PLGA 50/50 having a lower average molecular weight, showed a mean particle size of 87.1 μm.

Batch 69 obtained using a reactor with a flat bottom in steps 4 and 5, no sodium chloride in step 4, no step 7, showed a mean particle size of 56.5 μm and an entrapment efficiency of 70.6%.

### EXAMPLE 2

In Vitro Release Profile of Triptorelin Acetate Encapsulating Microparticles

The lyophilisate microparticles were put in a methanol/water mixture under stirring at 200 rpm at 37° C. in a test representative of the physiological conditions in the human body. Samples from this mixture were analyzed as a function of time by HPLC.

The in vitro release curves for the seven batches of Triptorelin acetate encapsulating microparticles are represented in FIGS. 1A and 1B.

Those curves show for all batches a release of the therapeutically active substance of less than 3% during the first 48 hours.

### EXAMPLE 3A

Effect of Injection of Triptorelin Acetate Encapsulating Microparticles in Rats on the Serum Triptorelin Acetate Levels

Protocol: 6 adult male rats are given an intramuscular injection of a suspension of Triptorelin acetate encapsulating microparticles in sterile distilled water. Blood samples are then collected regularly from day 1 (day of the injection) through day 35 for determining of Triptorelin acetate levels.

FIG. 2A represents the variation of the cum AUC (cumulated area under the curve) of the Triptorelin acetate level for batches 56 and 69 of Triptorelin acetate as a function of time.

That curve shows that the cum AUC, i.e. the burst, is less than 10% after 24 hours and the variation of that parameter is linear until day 35.

### EXAMPLE 3B

Effect of Injection of Triptorelin Acetate Encapsulating Microparticles in Rats on the Serum Testosterone Levels

6 adult male rats previously housed close to female rats (for testosterone stimulation) are given an intramuscular injection of a suspension of Triptorelin acetate encapsulating microparticles in sterile distilled water. Blood samples are then collected regularly from day 1 (day of the injection) through day 42 for determining of testosterone levels.

The curves of mean testosterone levels as a function of time (expressed in days) for Triptorelin acetate encapsulating microparticles of batches 56 and 57 and a control, are represented in FIG. 2C.

Those curves show that satisfying testosterone levels below 3.5 nmol/l corresponding to a castration condition are obtained for all samples as from day 5 to day 36.

### EXAMPLE 4

Preparation of Tamoxifen Encapsulating Microparticles

Batch 4 of Tamoxifen encapsulating microparticles was prepared using a sequence of steps similar to that described in Example 1 for batch 56, with the main difference that in the first step water-insoluble Tamoxifen is dissolved in the ethyl acetate solution together with the PLGA 50/50 having a average molecular weight of 45,000, the following steps being very similar to steps 4 to 8.

That batch showed a mean particle size of 49.6 μm as determined by laser granulometry and an encapsulation efficiency of 82.8%.

### EXAMPLE 5

In Vitro Release Profile of Tamoxifen Encapsulating Microparticles

The lyophilisate microparticles were put in a methanol/water mixture under stirring at 200 rpm at 37° C. in a test representative of the physiological conditions in the human body. Samples from this mixture were analyzed as a function of time by HPLC.

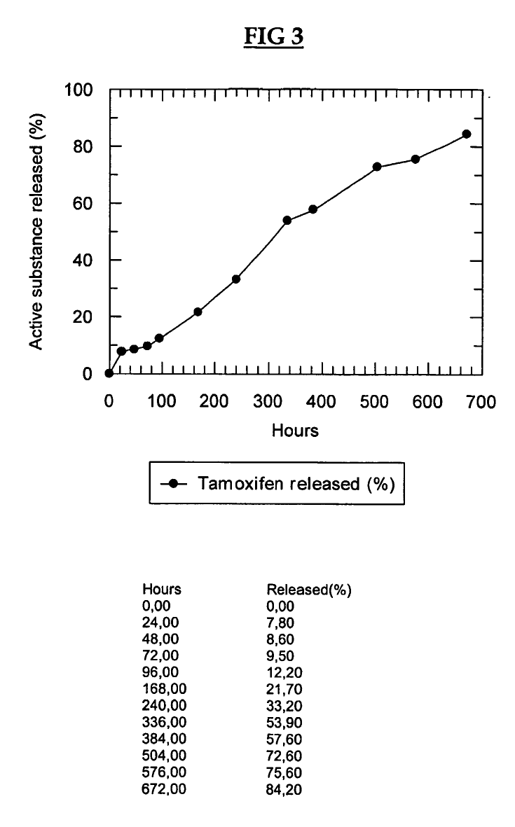

The in vitro release curve for batch 4 of Tamoxifen encapsulating microparticles is represented in FIG. 3.

That curve shows a release of the therapeutically active substance of less than 10% during the first 48 hours, and a linear release up to 1 month.

### EXAMPLE 6

Preparation of 4-OH-Tamoxifen Encapsulating Microparticles and In Vitro Release Profile thereof

Batch 5 of the Z isomer of 4-OH-Tamoxifen encapsulating microparticles was prepared using a sequence of steps similar to that described in Example 1 for batch 56, with the main difference that in the first step water-insoluble 4-OH-Tamoxifen is dissolved in the ethyl acetate solution together with the PLGA 50/50 having a average molecular weight of 45,000, the following steps being very similar to steps 4 to 8.

That batch showed a mean particle size of 53.98 μm as determined by laser granulometry and an encapsulation efficiency of 66.92% on the lyophilisate.

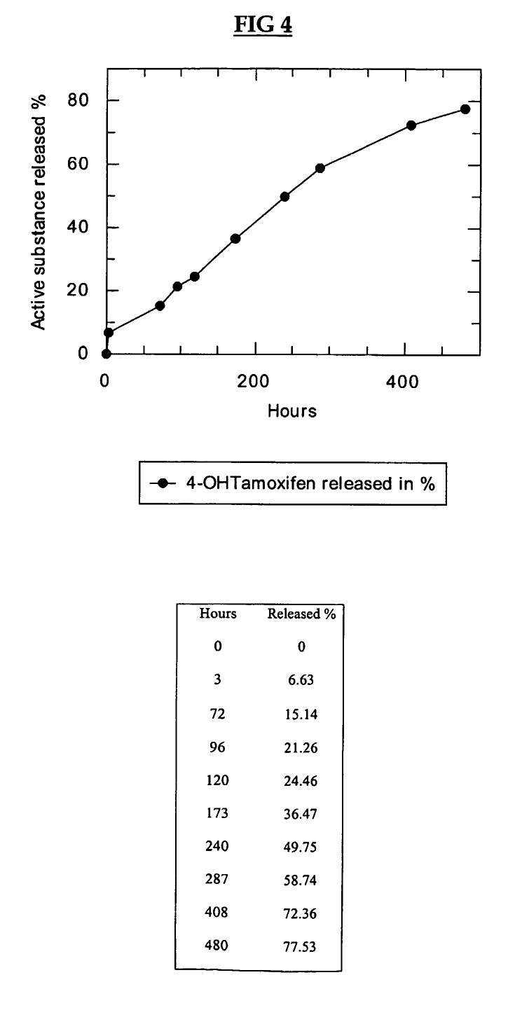

An in vitro release test similar to that described in Example 2 showed a release of the therapeutically active substance of about 9.2%, i.e. a burst of less than 10% during the first 24 hours, and a linear release up to 500 hours (see FIG. 4).

### EXAMPLE 7

Preparation of RC-160 Encapsulating Microparticles

The following sequences of steps was performed:

1. About 175.0 mg of Vapreotide acetate were dissolved in 2.0 g of sterile distilled water. This aqueous phase solution was cooled to 4° C.

2. About 5.0 g of poly(D-L-lactide-co-glycolide) (PLGA) with a ratio of lactide to glycolide of 50/50 and a weight average molecular weight of 45,000 Dalton were dissolved in 50.0 g of ethyl acetate at room temperature. This organic solution was cooled to 4° C.

3. The aqueous phase solution was poured into the organic phase solution and the mixture was homogenized using a Polytron PT 6100 (PT-DA 3020/2TM shaft) at 20,000 rpm during 2 minutes.

4. This w/o preparation was poured into about 1687.5 g of aqueous phase containing 20% (w/w) of polyoxyethylene sorbitan fatty acid ester (Tween 80) in a reactor kept at a temperature of 4° C.

5. The homogenization was performed using a Polytron PT 6100 (PT6060/2TM shaft) at 3,000 rpm during 5 minutes, thereby forming a suspension microparticles.

6. The microparticles were collected by filtration and washed with bout 1.7 l of sterile distilled water yielding a bulk of microparticles.

7. The bulk was possibly frozen, kept overnight and thawed.

8. The microparticles were suspended in a lyophilization medium consisting of mannitol and sodium carboxy-methyl-cellulose (and possibly Tween 80) using an IKA T25 homogenizes at 9,500 rpm during 30 minutes. The suspension was poured on a tray and freeze-dried.

9. The obtained freeze-dried microparticles were sieved on 106 μm.

The obtained freeze-dried microparticles showed less than 2% residual water. The entrapment efficiency was measured by HPLC on the freeze-dried microparticles and the particles size distribution was determined using a laser granulometer (Mastersizer™, Malvern Instruments).

An in vitro release test similar to that described in Example 2 showed a release of the therapeutically active substance of less than 10% during the first 24 hours, and a linear release up to 500 hours.

1. Microparticles of biodegradable polymer encapsulating a water-soluble or water-insoluble biologically active substance, wherein pocket microparticles contain microparticles of smaller size, said microparticles are obtainable by a method comprising
(c) pouring an organic liquid phase comprising, in a dissolved state the biodegradable polymer and in a uniformly distributed state the biologically active substance, in a non-water miscible organic solvent showing a low solubility in water, into an aqueous liquid phase of sufficient volume to dissolve said organic solvent, said aqueous phase containing a surfactant, and homogenizing the resulting organic/aqueous phase, thereby forming a suspension of microparticles, and
(d) filtering the suspension obtained in (a), optionally washing the microparticles with water, suspending the microparticles without vacuumdrying thereof in a lyopholisation medium and freeze-drying.

2. Microparticles of claim 1 which show no, or a very low burst when releasing said active substance.

3. Microparticles of claim 2 wherein the initial release of the active substance is less than 10% during the first 24 hours.

4. Microparticles of claim 2 wherein the initial release of the active substance is less than 3% during the first 48 hours.

5. Microparticles according to any of claims 1 to 4 wherein the biodegradable polymer is a poly(D-L-lactide-co-glycolide).

6. Microparticles according to any of the preceding claims wherein the biologically active substance is a water-soluble substance selected from peptide, a polypeptide, a protein and the related pharmaceutically acceptable salts thereof.

7. Microparticles according to claim 6 wherein the biologically active substance is a luteinizing hormone releasing hormone (LHRH) or a derivative thereof, in particular Triptorelin acetate.

8. Microparticles according to any of claims 1 to 5 wherein the biologically active substance is a water-insoluble substance selected from Tamoxiphen, 4-OH Tamoxiphen, a derivative thereof, a water-insoluble LHRH derivative such as Triptorelin pamoate and a water-insoluble somatostatin derivative such as vapreotide pamoate.

9. Microparticles according to any of previous claims wherein the organic solvent of step (a) is ethyl acetate.

10. Microparticles according to any of previous claims wherein in step (a) the volume ratio of the organic liquid phase to the aqueous liquid phase is comprised between 0.007 and 0.06.

11. Microparticles according to claim 9 or 10 wherein the temperature of the organic phase is comprised between 2° C. and 8° C., preferably between 3 and 5° C.

12. Microparticles according to any of the previous claims wherein in step (a) the surfactant is Tween 80.

13. A sustained release pharmaceutical formulation which comprises a suspension of microparticles according to any of claims 1 to 12 in a pharmaceutically acceptable vehicle.

14. A method of preparing microparticles according to any of claims 1 to 12 comprising
(a) pouring an organic liquid phase comprising, in a dissolved state the biodegradable polymer and in a uniformly distributed state the biologically active substance, in a non-water miscible organic solvent showing a low solubility in water, into an aqueous liquid phase of sufficient volume to dissolve said organic solvent, said aqueous phase containing a surfactant, and homogenizing the resulting organic/aqueous phase, thereby forming a suspension of microparticles, and
(b) filtering the suspension obtained in (a), optionally washing the microparticles with water, suspending the microparticles without vacuum-drying thereof in a lyophilisation medium and freeze-drying.
